(19) United States
(12) Patent Application Publication
(10) Pub. No.: US 2025/0082572 A1
(43) Pub. Date: Mar. 13, 2025

(54) TOPICAL FORMULATIONS FOR HANDLING ATOPIC DERMATITIS

(71) Applicant: GOLD FEET LTD, Haifa (IL)

(72) Inventor: Arie WALDMAN, Haifa (IL)

(21) Appl. No.: 18/723,046

(22) PCT Filed: Dec. 22, 2022
(86) PCT No.: PCT/IL2022/051371

§ 371 Date: Jun. 21, 2024

(30) Foreign Application Priority Data
Dec. 26, 2021 (IL) 289401

(51) Int. Cl. A61K 9/06; A61K 9/00; A61K 31/573; A61K 45/06; A61K 47/44; A61K 47/46; A61P 17/00

(52) U.S. Cl. CPC A61K 9/06; A61K 9/0014; A61K 31/573; A61K 45/06; A61K 47/44; A61K 47/46; A61P 17/00

(57) ABSTRACT

A topical composition for use in treating dermatitis, comprising from 0.5 to 5 wt % olive oil, from 0.3 to 3 wt % date honey, from 0.3 to 3 wt % coconut oil, and a steroid anti-inflammatory agent in an amount of from 0.5 ppm to 50 ppm, homogeneously admixed in a pharmaceutically acceptable cream. A method of making the composition is also disclosed.

### RELATED APPLICATIONS

This PCT application claims the benefit according to 35 U.S.C. § 119 (a) of Israeli patent application 289401 filed on Dec. 26, 2021 and having the same title and applicant as the present application; and this earlier application is fully incorporated herein by reference.

### FIELD OF THE INVENTION

The present invention relates to compositions for mitigating dermatitis, particularly to compositions for handling atopic dermatitis, more particularly to topical compositions for treating atopic dermatitis that may be employed on large skin areas for prolonged periods of time, even in children.

### BACKGROUND OF THE INVENTION

Atopic dermatitis (AD) is an immune-mediated skin inflammation associated with pruritus and intensive itching on the afflicted body surface, usually including face, neck, scalp, extremities, and abdomen, particularly on flexural surfaces. AD afflicts mainly children, but exacerbations are common into adulthood. The debilitating condition affects up to 20% children and up to 10% adults in developed countries. Even if the disease later resolves, AD may leave long-term psychologic sequelae as the children are challenged with visible skin changes during formative years (The Merck Manual of Diagnosis and Therapy, 19th Edition, 2011).

Beside employing supportive care, AD has been mainly handled by applying topical corticosteroids, the ointments usually containing between 0.05 and 0.5 wt % corticosteroids, wherein the treatment regimen may include as much as 300 grams ointment for a 10-days course. However, high corticosteroid quantities, large treated areas, or long-term corticosteroid use, result in local adverse effects including striae, petechiae, telangiectasia, skin thinning, atrophy, and worsening acne, or in systemic adverse effects including hypothalamic-pituitary-adrenal axis suppression, reduced linear growth in children, and bone density changes in adults. It is therefore an object of the present invention to provide a topical means for mitigating the symptoms of AD without the drawbacks of the known treatments.

It is a further object of this invention to provide a topical composition comprising a steroid anti-inflammatory agent, the composition essentially lacking known adverse effects even if employed on large body areas for prolonged periods.

It is still another object of this invention to provide a composition for mitigating or eliminating the symptoms of atopic dermatitis, the composition employing naturally occurring plant materials, while avoiding usually employed therapeutic amounts of corticosteroids.

The invention aims at providing a method for preparing a safe and efficient composition for handling skin inflammation, the composition employing naturally occurring plant materials, while avoiding usually employed therapeutic amounts of corticosteroids.

Other objects and advantages of the present invention will appear as the description proceeds.

### SUMMARY OF THE INVENTION

This invention provides a topical composition for use in treating dermatitis, comprising from 0.5 to 5 wt % olive oil, from 0.3 to 3 wt % date honey, from 0.3 to 3 wt % coconut oil, and a steroid anti-inflammatory agent in an amount of from 0.5 ppm to 50 ppm, homogeneously admixed in a pharmaceutically acceptable cream. The symbol “wt %” means weight percent. Said steroid may be selected from the group consisting of hydrocortisone, fluocinolone acetonide, alclometasone dipropionate, desonide, hydrocortisone butyrate, fluticasone propionate, betamethasone valerate, betamethasone dipropionate, betamethasone valerate, fluticasone propionate, fluocinonide, desoximetasone, betamethasone dipropionate, halobetasol propionate, and clobetasol propionate. The composition is preferably employed for mitigating the symptoms of or for treating atopic dermatitis. Said acceptable cream is preferably an oil in water emulsion comprising paraffin. The composition is unique in employing a very low concentration of steroid anti-inflammatory agent, being between 0.5 and 50 ppm, preferably between 1 and 20 ppm, so that large body areas may be treated, even in small children, and for prolonged periods of time. As other dermatoses are also treated with topical steroid means, a person skilled in the art will advantageously employ the composition of the invention also for them, when possible. In one preferred embodiment of the invention, 5 to 15 ppm clobetasol propionate is employed in said topical composition, advantageously for atopic dermatitis. The composition of the invention may further comprise pharmaceutically acceptable carriers, excipients, diluents, or surfactants, as well as additional active agent(s) selected from antiviral, antibacterial, antifungal, antibiotic, antiseptic, antihistamine, analgesic, anti-inflammatory, immunomodulatory, or a material adjusting biological tissue permeability.

The invention is directed to a process for manufacturing a topical composition, preferably a skin cream, comprising homogenizing, at a temperature between ambient and 100° C., from 0.5 to 5 wt % olive oil, from 0.3 to 3 wt % date honey, from 0.3 to 3 wt % coconut oil, and a steroid anti-inflammatory agent in an amount of from 0.5 ppm to 50 ppm in an oil-in-water emulsion comprising paraffin, optionally together with additives or additional active agents.

### DETAILED DESCRIPTION OF THE INVENTION

It has now been found that atopic dermatitis may be efficiently treated on large body surfaces, even in children, and for prolonged periods of time with topical steroids without side effects, when employing a cream composition comprising steroids(S) together with date honey (D), olive oil (O), and coconut oil (C), wherein said steroid may be present in an amount of 10 ppm or 5 ppm (the compositions denoted below as SDOC10 or SDOC5).

It is well known that corticosteroids may have serious side effects even when used topically and therefore, to minimize their toxicity, the shortest duration needed to control the flare-up is recommended. However, this may often pose a difficult limitation, particularly in resistant or complicated cases, or in case of young children. The invention will enable to prolong the treatment periods and to more efficiently and flexibly organize the whole regimen, while optimizing preventive strategies and substantially reducing the risks of the side effects.

In a preferred embodiment, the components in accordance with the invention, namely the naturally obtained plant products of date honey, olive oil and coconut oil are admixed in a topical composition together with a minimal and nearly harmless-even though surprisingly effective-amount of the steroid active agent, such as clobetasol propionate, the composition being a pharmaceutically acceptable ointment or cream, preferably cream comprising paraffin. The composition is manufactured by known methods for preparing cosmetic or pharmaceutical creams. Without wishing to be limited by any particular theory, the inventors believe that the combination of said plant products and the cream components employed in the composition improves the steroid utilization and effectiveness on the skin surface possibly by increasing its thermodynamic activity.

The invention relates to a plant-based means combined with anti-inflammatory steroids in ppm concentrations for mitigating skin inflammatory conditions, particularly atopic dermatitis. The means includes topical compositions comprising mentioned agents and possibly pharmaceutically acceptable carrier, excipient, diluent, or other additive; additives may comprise coloring agents, preservatives, fillers, thickeners, solvents, materials modifying visual or olfactory properties, and materials adjusting viscosity, density or biological tissue permeability. The compositions for topical use of the invention may have a consistency of cream, lotion, ointment, but preferably it is a cream, in a preferred embodiment paraffin comprising cream. In particular cases, it is preferable to include an additional active agent in the composition, which may act independently or synergistically, for example selected from antiviral, antibacterial, antifungal, antibiotic, antiseptic, antihistamine, anti-inflammatory, or immunomodulator. For example, said antibacterial agent may assist in suppressing streptococcal or staphylococcal secondary infections, or said antiviral, such as acyclovir, may suppress eventual herpetic eruption.

AD is often hard to differentiate form other dermatoses, such as from seborrheic dermatosis, contact dermatitis, nummular dermatitis, and psoriasis; the composition of the invention mat be very useful even in handling said dermatoses.

The invention will be further described and illustrated by the following examples.

### EXAMPLES

### Materials

Aquosum cream (Water, Petrolatum, Mineral oil, Cetearyl alcohol, paraffin, Glyceryl stearate, PEG-100 stearate, Tocoppheryl acetate, Sodium Lauryl Sulfate, P-Chloro-m-cresol) was obtained commercially. Johnson and Johnson, Pfizer and Bombastus-Werk all produce Aquosum cream which is widely available.

Clobetasole propionate cream 0.05% (W/W) was obtained from Glaxo-SmithKline under the trade name DERMOVATE. The active ingredient is dispersed in a carrier comprising propylene glycol, sorbitan sesquoleate and white soft paraffin.

Olive oil was obtained from Kermey Golan (Israel) as “Barnea Dominant” oil with 0.4% acidity maximum.

Coconut oil was obtained from Cocomi Bio-Organic (Sri Lanka).

Date honey (Silan) was obtained from Meshek Line (Israel).

### Example 1

### Preparing Anti-Dermatitis Cream

Clobetasol propionate cream 0.05%, 25 g, was heated to 80° C. for 3 minutes and in molten state mixed with 1000 g Aquosum cream in a mixer. Olive oil, 33 ml, was admixed to the mixed creams and homogenized in the mixer. Coconut oil, 16.6 ml, was heated to 80° C. for 3 minutes and mixed with the cream in the mixer. Date honey, 16.6 ml, was heated to 80° C. for 3 minutes, and the liquid was passed through a textile filter into the cream in the mixer. The mixture was well homogenized to provide a final cream product according to one embodiment of the invention. The components contents of the anti-dermatitis cream according to this embodiment are summarized in Table 1. The cream according to the invention, comprising Steroid/Date/Olive/Coconut, is denoted SDOC, and when containing 0.0011% steroid, which corresponds to 11 ppm, the composition is denoted as “SDOC11”.

TABLE 1
Anti-dermatitis cream SDOC11 according
to one embodiment of the invention

| Ingredient | Amount | Final concentration |
|---|---|---|
| Steroid | 12.5 mg clobetasol propionate | 0.0011 wt % Steroid = |
| | in 25 g carrier cream | 11 ppm |
| Date honey | 16.6 ml (about 18.2 g) | about 1.7 wt % Date |
| Olive oil | 33 ml (about 30.2 g) | about 3 wt % Olive |
| Coconut oil | 16.6 ml (about 15.3 g) | about 1.4 wt % Coconut |
| Aquosum and | 1025 g (paraffin in water) | about 94 wt % |
| carrier creams | | |

In some tests, Clobetasol propionate cream 0.05%, 15 g was added to the components as defined above, providing the final clobetasol propionate concentration of 0.00066% (=6.6 ppm, about 7 ppm), the composition being denoted as SDOC7.

### Example 2

### Treating Atopic Dermatitis

Twenty volunteers, 10-17 years old, showing history of atopic dermatitis (AD) and exhibiting symptoms of acute AD, including pruritus on neck, hands, parts of abdomen, and flexural areas opposite to the elbow and kneecap, were administered topical treatment with 15 g composition SDOC11 twice a day. In all cases, the itching symptoms were alleviated within 1 to 4 days, and the pruritus disappeared within 2 to 7 days. The treatment was continued for 14 days, and all volunteers remained free of any AD symptoms during the follow up period of 2 weeks to 2 months.

### Example 3

### Treating Atopic Dermatitis

Ten volunteer, 18-25 years old, showing history of atopic dermatitis (AD) and exhibiting symptoms of AD, including pruritus on neck, hands, and flexural areas opposite to the elbow and kneecap, were administered topical treatment with 15 g composition SDOC7 twice a day. In all cases, the itching symptoms were alleviated within 1 to 3 days, and the pruritus disappeared within 2 to 6 days. The treatment was continued for 10 days, and all volunteers remained free of any AD symptoms during the follow up period of of 2 weeks to 2 months.

While the invention has been described using some specific examples, many modifications and variations are possible. It is therefore understood that the invention is not intended to be limited in any way, other than by the scope of the appended claims.

1. A topical composition for use in treating dermatitis, comprising from 0.5 to 5 wt % olive oil, from 0.3 to 3 wt % date honey, from 0.3 to 3 wt % coconut oil, and a steroid anti-inflammatory agent in an amount of from 0.5 ppm to 50 ppm, homogeneously admixed in a pharmaceutically acceptable cream.

2. The composition of claim 1, wherein said steroid is selected from the group consisting of hydrocortisone, fluocinolone acetonide, alclometasone dipropionate, desonide, hydrocortisone butyrate, fluticasone propionate, betamethasone valerate, betamethasone dipropionate, betamethasone valerate, fluticasone propionate, fluocinonide, desoximetasone, betamethasone dipropionate, halobetasol propionate, and clobetasol propionate.

3. The composition of claim 1, wherein said dermatitis is atopic dermatitis.

4. The composition of claim 1, wherein said cream is an oil in water emulsion comprising paraffin.

5. The composition of claim 4, wherein oil in water emulsion comprising paraffin is aquosum cream.

6. The composition of claim 1 for use in treating atopic dermatitis, comprising from 1 to 20 ppm clobetasol propionate.

7. The composition of claim 1 for use in treating atopic dermatitis, comprising from 5 to 15 ppm clobetasol propionate.

8. The composition of claim 1, further comprising pharmaceutically acceptable carriers, excipients, or diluents.

9. The composition of claim 1, further comprising an additional active agent selected from antiviral, antibacterial, antifungal, antibiotic, antiseptic, antihistamine, analgesic, anti-inflammatory, immunomodulatory, or a material adjusting biological tissue permeability.

10. A process for manufacturing the composition of claim 1, comprising homogenizing from 0.5 to 5 wt % olive oil, from 0.3 to 3 wt % date honey, from 0.3 to 3 wt % coconut oil, a steroid anti-inflammatory agent in an amount of from 0.5 ppm to 50 ppm, optionally additives and additional active agents, in an oil-in-water emulsion comprising paraffin, at a temperature from ambient to 100° C.
